(19) United States
(12) Patent Application Publication
(10) Pub. No.: US 2014/0079789 A1
(43) Pub. Date: Mar. 20, 2014

(54) Bioglass with Glycosaminoglycans

(71) Applicants: Gregory J. POMRINK, Newberry, FL (US); Zehra TOSUN, Gainesville, FL (US)

(72) Inventors: Gregory J. POMRINK, Newberry, FL (US); Zehra TOSUN, Gainesville, FL (US)

(73) Assignee: NOVABONE PRODUCTS, LLC, Alachua, FL (US)

(21) Appl. No.: 14/030,140

(22) Filed: Sep. 18, 2013

Related U.S. Application Data
(60) Provisional application No. 61/702,445, filed on Sep. 18, 2012.

(51) Int. Cl. A61K 47/48; A61K 33/08

(52) U.S. Cl. CPC A61K 47/4823; A61K 33/08
USPC 424/493; 424/692; 536/123.1

(57) ABSTRACT

One or more particles of bioactive glass coated with a glycosaminoglycan, wherein the glycosaminoglycan is bound to the bioactive glass and methods of treatment using such particles.

### CROSS-REFERENCE TO RELATED APPLICATIONS

This application claims the benefit of U.S. Provisional Application No. 61/702,445, filed Sep. 18, 2012, the entire contents of which are hereby incorporated herein by reference. This application further claims the benefit of PCT Application No. PCT/US2013/060280, filed Sep. 18, 2013, the entire contents of which are hereby incorporated by reference.

### BACKGROUND OF THE INVENTION

Bone injuries do not often occur in isolation. Often there is significant wounding and trauma to the soft tissue surrounding the injured bone. A bone fracture is often associated with significant bruising of the surrounding tissue. It may be the case that delayed wound healing may serve to delay repair of the fractured bone, such as by promoting excessive and long-lasting inflammation. Thus, there is a need to combine treatments and agents promoting bone repair, such as bioactive glass, with a therapeutic agent that promotes soft tissue wound healing.

Bioactive glass was originally developed in 1969 by L. Hench. Additionally bioactive glasses were developed as bone replacement materials, with studies showing that bioactive glass can induce or aid in osteogenesis. Hench et al, J. Biomed. Mater. Res. 5:117-141 (1971). Bioactive glass can form strong and stable bonds with bone. Piotrowski et al., J. Biomed. Mater. Res. 9:47-61 (1975). Further, bioactive glass is not considered toxic to bone or soft tissue from studies of in vitro and in vivo models. Wilson et al., J. Biomed. Mater. Res. 805-817 (1981). Exemplary bioactive glasses known in the art include 45S5, 45S5B1, 58S, and 570C30. The original bioactive glass, 45S5, is melt-derived. Sol-gel derived glasses have nanopores that allow for increased surface area and bioactivity. However, bioactive glass may not be sufficient for ensuring that both damaged bone and the associated wounded soft tissue are both repaired. Bioactive glass may also promote coagulation of blood, which would impair the healing of a soft tissue wound.

With regard to soft tissue, glycosaminoglycans (GAGs) play a key role in promoting healing and/or reducing coagulation. GAGs are polysaccharides found in humans and animals, with several located in the tissues and fluids. The known GAGs are chondroitin sulfate, keratan sulfate, dermatan sulfate, hyaluronic acid, heparin, and heparan sulfate. As with collagen, GAGs are a major provider of structure to animal tissues. They are required for the repair and treatment of wounded tissue.

GAGs have been included with other materials in the preparation of wound healing prosthetics. U.S. Pat. No. 5,166,187 discloses a biomaterial consisting of an association of collagen, chitosan acetylated to a degree of acetylation between about 10% and about 40% and of glycosaminoglycans. The disclosed biomaterial is used for making extracellular matrices for regeneration of nerve cells and bones as well as biocompatible envelopes. A particular application is the making of artificial skin consisting of a dermal layer.

U.S. Patent App. No. 2007/0134285 to Lynn et al. discloses a material comprising collagen, brushite, and one or more glycosaminoglycans found in an aqueous solution. Mixtures of GAGs with other materials are disclosed in U.S. Pat. No. 7,887,831 to Yayon. Also, bioactive glass treated with chitosan is disclosed in Oudadesse, H. et al., “Chitosan Effects on Bioactive Glass for Application as Biocomposite Biomaterial” Intl. J. Biol. Biomed. Eng. 2011, 2(5):49-56.

Applicant has discovered that GAGs, when coated on bioactive glass, are still able to promote soft-tissue wound healing without significantly interfering with the activity of the bioactive glass on bone repair. Coating bioactive glass with GAGs provides a solution to the problem of reducing the unwanted coagulation that arises from the presence of bioactive glass.

### BRIEF SUMMARY OF THE INVENTION

One aspect of the invention provides for bioactive glass coated with a glycosaminoglycan. The glycosaminoglycan is bound to the bioactive glass. The bioactive glass may be in the form of a particle, a glass sheet, a fiber, a mesh, or any combination of these forms.

The invention also provides, in another aspect, for a material comprising collagen and bioactive glass, with the bioactive glass coated with a glycosaminoglycan. The glycosaminoglycan is bound to the bioactive glass. The bioactive glass may be in the form of a particle, a glass sheet, a fiber, a mesh, or any combination of these forms.

Another aspect of the invention provides for a method for treating a wound. Bioactive glass coated with a glycosaminoglycan is applied to the wound. The bioactive glass may be in the form of a particle, a glass sheet, a fiber, a mesh, or any combination of these forms. The wound comprises one or more of a bone injury and a soft tissue injury. The coated bioactive glass is effective to accelerate repair of the bone injury and the soft tissue injury.

Another aspect of the invention provides for a method of treating a bone defect. A bioactive glass coated with a glycosaminoglycan is applied to the site at or near the bone defect. The bioactive glass may be in the form of a particle, a glass sheet, a fiber, a mesh, or any combination of these forms. The coated bioactive glass is bioresorbable at a rate consistent with the rate of formation of new bone at or near the site.

Another aspect of the invention provides for a method of stimulating the activity of a gene that promotes wound healing and/or bone regeneration. Bioactive glass coated with a glycosaminoglycan is applied to the site at or near the bone defect. The bioactive glass may be in the form of a particle, a glass sheet, a fiber, a mesh, or any combination of these forms. The activity of the gene is stimulated.

Another aspect of the invention provides for a method of reducing inflammation at or near the site of a wound and/or a bone defect. Bioactive glass coated with a glycosaminoglycan is applied to the site of the wound or at or near the bone defect.

Another aspect of the invention provides for a method of preparing a composition comprising bioactive glass coated with a glycosaminoglycan. The glycosaminoglycan is applied to the bioactive glass by means of dipping or spraying. The bioactive glass with glycosaminoglycan is then freeze-dried such that the glycosaminoglycan becomes attached to the bioactive glass by a physical bond.

Yet another aspect of the invention provides for a method of preparing a composition comprising bioactive glass coated with a glycosaminoglycan. A sol-gel solution comprising GAGs is electrospinned. The electrospinning may be performed in the presence of bioactive glass particles dispersed within the solution. The bioactive glass may be in the form of an interpenetrating network of bioglass.

### BRIEF DESCRIPTION OF THE DRAWINGS

FIG. 1 depicts the rate of dissolution of putty made in accordance with the description by sterilization technique over time.

FIG. 2 includes photomicrographs of putty made in accordance with the description by sterilization technique at time intervals.

### DETAILED DESCRIPTION OF THE INVENTION

One aspect of the invention provides for bioactive glass coated with a glycosaminoglycan. The glycosaminoglycan is bound to the bioactive glass. The bioactive glass may be in the form of a particle, a glass sheet, a fiber, a mesh, or any combination of these forms.

In this and other aspects of the invention, the glycosaminoglycan may be present in amounts of 1-3 wt. % ratio with reference to the total weight of the bioactive glass coated with glycosaminoglycan. The glycosaminoglycan may also be present in a weight ratio of up to 5 wt. %, 10 wt. %, 15 wt. %, 20 wt. %, and 25 wt. %, as well as in a 2-4 wt. % ratio, 3-5 wt. % ratio, 4-6 wt. % ratio, 5-10 wt. % ratio, 10-15 wt. % ratio, 15-20 wt. % ratio, or 20-25 wt. % ratio. Alternatively, the weight ratio may be from 1-1.5%, 1.1-1.6%, 1.2-1.7%, 1.3-1.8%, 1.4-1.9%, 1.5-2.0%, 1.6-2.1%, 1.7-2.2%, 1.8-2.3%, 1.9-2.4%, 2.0-2.5%, 2.1-2.6%, 2.2-2.7%, 2.3-2.8%, 2.4-2.9%, or 2.5-3.0%. In some embodiments of this and other aspects of the invention, the weight ratio may be about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, or about 3.0%. In some embodiments of this and other aspects of the invention, the weight ratio may be 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%. When making a putty, the glycosaminoglycan may be present in an amount of about 2.5% wt % ratio or greater to ensure the putty is moldable and irrigation resistant. When preparing crosslinked sheets, sponges, and composites amounts of glycosaminoglycan less than about 2.5 wt % may also be used.

Bioactive glass used in the invention may be melt-derived or sol-gel derived. Depending on their composition, bioactive glasses of the invention may bind to soft tissues, hard tissues, or both soft and hard tissues. The composition of the bioactive glass may be adjusted to modulate the degree of bioactivity. Furthermore, borate may be added to bioactive glass to control the rate of degradation. Additional elements, such as copper, zinc, and strontium may be added to bioactive glass to facilitate healthy bone growth. Bioactive glass that may also be suitable include glasses having about 40 to about 60 wt % SiO₂, about 10 to about 34 wt % Na₂O, up to about 20 wt % K₂O, up to about 5 wt % MgO, about 10 to about 35 wt % CaO, 0 to about 35 wt % SrO, up to about 20 wt % B₂O₃, and/or about 0.5 to about 12 wt % P₂O₅. The bioactive glass may additionally contain up to 10 wt % CaF₂.

Bioactive glass is capable of bonding to bone, which begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer is another layer which becomes increasingly silica rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

Application of bioactive glass to bone may promote bone remodeling. Bone remodeling occurs by equilibrium between osteoblast-mediated bone formation and osteoclast-mediated bone destruction. When bone is injured or missing, such as in a fracture, promotion of osteoblast activity is thought to be helpful to induce bone formation. Further, promoting bone formation by osteoblasts may be helpful in locations in which there is significant bone loss in the absence of an apparent injury.

The bioactive glass may have osteostimulative properties, which refers to promoting proliferation of the osteoblasts such that bone can regenerate. In an osteostimulative process, a bioactive glass material may be colonized by osteogenic stem cells. This may lead to quicker filling of bone defects than would otherwise occur with an osteoconductive glass.

In various embodiments of this and other aspects of the invention, the bioactive glass sheets, fibers, and mesh may provide structure to a tissue site in order to support, promote or facilitate new tissue growth.

The bonding of bioactive glass to bone begins with the exposure of bioactive glass to aqueous solutions. Sodium ions in the glass can exchange with hydronium ions in body fluids, which increases the pH. Calcium and phosphorous ions can migrate from the glass to form a calcium and phosphate-rich surface layer. Borate ions can also migrate from the glass to from a surface layer rich in boron. Strontium ions also can migrate from the glass to form a strontium-rich surface layer. Underlying this surface layer of the bioactive glass is another layer which becomes increasingly silica rich due to the loss of sodium, calcium, strontium, boron, and/or phosphate ions (U.S. Pat. No. 4,851,046). Hydrolysis may then disrupt the Si—O—Si bridges in the silica layer to form silanol groups, which can disrupt the glass network. The glass network is then thought to form a gel in which calcium phosphate from the surface layer accumulates. Mineralization may then occur as calcium phosphate becomes crystalline hydroxyapatite, which effectively mimics the mineral layer of bones.

Bioactive glass particles, fibers, meshes or sheets may be prepared by a sol-gel method. Methods of preparing such bioactive active glasses are described in Pereira, M. et al., “Bioactive glass and hybrid scaffolds prepared by sol-gel method for bone tissue engineering” Advances in Applied Ceramics, 2005, 104(1): 35-42 and in Chen, Q. et al., “A new sol-gel process for producing Na₂O-containing bioactive glass ceramics” Acta Biomaterialia, 2010, 6(10):4143-4153.

The composition can be allowed to solidify. In some embodiments, particles of bioactive glass are sintered to form a porous glass.

Repeated cooling and reheating may be performed on the solidified or sintered bioactive glass, with or without spinning, to draw the bioactive glass produced into fibers. A glass drawing apparatus may be coupled to the spinner and the source of molten bioactive glass, such as molten bioactive glass present in a crucible, for the formation of bioactive glass fibers. The individual fibers can then be joined to one another, such as by use of an adhesive, to form a mesh. Alternatively, the bioactive glass in molten form may be placed in a cast or mold to form a sheet or another desired shape.

The bioactive glass particles, fibers, meshes or sheets may further comprise any one or more of adhesives, grafted bone tissue, in vitro-generated bone tissue, collagen, calcium phosphate, stabilizers, antibiotics, antibacterial agents, antimicrobials, drugs, pigments, X-ray contrast media, fillers, and other materials that facilitate grafting of bioactive glass to bone.

A bioactive glass ceramic material suitable for the present compositions and methods may have silica, sodium, calcium, strontium, phosphorous, and boron present, as well as combinations thereof. In some embodiments, sodium, boron, strontium, and calcium may each be present in the compositions in an amount of about 1% to about 99%, based on the weight of the bioactive glass ceramic. In further embodiments, sodium, boron, strontium and calcium may each be present in the composition in about 1%, about 2%, about 3%, about 4%, about 5%, about 6%, about 7%, about 8%, about 9%, or about 10%. In certain embodiments, silica, sodium, boron, and calcium may each be present in the composition in about 5 to about 10%, about 10 to about 15%, about 15 to about 20%, about 20 to about 25%, about 25 to about 30%, about 30 to about 35%, about 35 to about 40%, about 40 to about 45%, about 45 to about 50%, about 50 to about 55%, about 55 to about 60%, about 60 to about 65%, about 65 to about 70%, about 70 to about 75%, about 75 to about 80%, about 80 to about 85%, about 85 to about 90%, about 90 to about 95%, or about 95 to about 99%. Some embodiments may contain substantially one or two of sodium, calcium, strontium, and boron with only traces of the other(s). The term “about” as it relates to the amount of calcium phosphate present in the composition means +/−0.5%. Thus, about 5% means 5+/−0.5%.

The bioactive glass materials may further comprise one or more of a silicate, borosilicate, borate, strontium, or calcium, including SrO, CaO, P₂O₅, SiO₂, and B₂O₃. An exemplary bioactive glass is 45S5, which includes 46.1 mol % SiO₂, 26.9 mol % CaO, 24.4 mol % Na₂O and 2.5 mol % P₂O₅. An exemplary borate bioactive glass is 45S5B1, in which the SiO₂ of 45S5 bioactive glass is replaced by B₂O₃. Other exemplary bioactive glasses include 58S, which includes 60 mol % SiO₂, 36 mol % CaO and 4 mol % P₂O₅, and S70C30, which includes 70 mol % SiO₂ and 30 mol % CaO. In any of these or other bioactive glass materials of the invention, SrO may be substituted for CaO.

The following composition, having a weight % of each element in oxide form in the range indicated, will provide one of several bioactive glass compositions that may be used to form a bioactive glass ceramic:

| | |
|---|---|
| SiO₂ | 0-86 |
| CaO | 4-35 |
| Na₂O | 0-35 |
| P₂O₅ | 2-15 |
| CaF₂ | 0-25 |
| B₂O₃ | 0-75 |
| K₂O | 0-8 |
| MgO | 0-5 |
| CaF | 0-35 |

The bioactive glass ceramic can be in the form of a three-dimensional compressible body of loose glass-based fibers in which the fibers comprise one or more glass-formers selected from the group consisting of P₂O₅, SiO₂, and B₂O₃. Some of the fibers have a diameter between about 100 nm and about 10,000 nm, and a length:width aspect ratio of at least about 10. The pH of the bioactive glass can be adjusted as-needed.

In some embodiments, the body comprises fibers having a diameter between about 100 nm and about 10,000 nm. The especially small diameter of these fibers renders them highly flexible so they form into the compressible body without breaking. In some embodiments the body includes fibers meeting these dimensional requirements in addition to other glass morphologies, such as fibers of other dimensions, microspheres, particles, ribbons, flakes or the like. The fibers may have a variety of cross section shapes, such as flat, circular, oval, or non-circular.

Bioactive glass ceramics may be prepared by heating a composition comprising one or more of SiO₂, CaH(PO₄), CaO, P₂O, Na₂O, CaCO₃, Na₂CO₃, K₂CO₃, MgO, and H₂BO₃ to a temperature between 1300 and 1500° C. such that the composition may form molten glass. An exemplary composition that can form fibers includes 40-60% SiO₂, 10-20% CaO, 0-4% P₂O₅, and 19-30% NaO. Other exemplary compositions include 45S5, which includes 46.1 mol % SiO₂, 26.9 mol % Ca), 24.4 mol % Na₂O and 2.5 mol % P₂O₅; 45S5B1, which includes 46.1 mol % B₂O₃, 26.9 mol % Ca), 24.4 mol % Na₂O and 2.5 mol % P₂O₅; 58S, which includes 60 mol % SiO₂, 36 mol % CaO and 4 mol % P₂O₅; and S70C30, which includes 70 mol % SiO₂ and 30 mol % CaO. Another exemplary composition includes 40 mol % SiO₂, 40 mol % B₂O₃, 20 mol % CaO, and 20 mol % Na₂O.

In some embodiments, the bioactive glass material (e.g., a particle, sheet, fiber, or mesh) is treated with certain buffer solutions to prepare the surface of the bioactive glass material for cell adhesion and controls pH prior to the exposure of the particles with cells. In some embodiments, the bioactive glass material is treated with the buffer solution or solutions before GAGs are bound to the material. In this context, the bioactivity and bone formation using the glass, fibers, mesh, or ceramic of the present invention may be enhanced by treating these with a buffer solution. The glass, fibers, mesh, or ceramic may be buffer-treated and dried before addition of the GAG.

In certain embodiments, the pre-treatment buffer solution has a starting pH of from about 6 to about 12 but may reach an end pH of about 9.5. Examples of buffers that might be suitable for the pre-treatment of the present invention include mixed sodium phosphate salts (such as Sorensen's Phosphate buffer, Millonig's Phosphate buffer, Karlsson and Shultz Phosphate buffer, Maunsbach Phosphate buffer, and Phosphate Buffered Saline (PBS); buffer pH of about 6.4-8.0), TAPS (3-{[tris(hydroxymethyl)methyl]amino}propanesulfonic acid; buffer pH of about 7.7-9.1), Bicine (N,N-bis(2-hydroxyethyl)glycine; buffer pH of 7.6-9.0), Tricine (N-tris(hydroxymethyl)methylglycine; buffer pH about 7.4-8.8), Tris (tris(hydroxymethyl)methylamine; buffer pH of about 7.5-9.0), HEPES (4-2-hydroxyethyl-1-piperazineethanesulfonic acid; buffer pH of about 6.8-8.2), TES (2-{[tris(hydroxymethyl)methyl]amino}ethanesulfonic acid; buffer pH of about 6.8-8.2), MOPS (3-(N-morpholino)propanesulfonic acid; buffer pH of about 6.5-7.9), PIPES (piperazine-N,N′-bis(2-ethanesulfonic acid); buffer pH of about 6.1-7.5), Cacodylate (dimethylarsinic acid; buffer pH of about 5.0-7.5), SSC (saline sodium citrate; buffer pH of about 6.5-7.5), or MES (2-(N-morpholino)ethanesulfonic acid; buffer pH of about 5.5-6.7). Any other buffer having appropriate pH buffering range of about 6 to about 12 might be suitable.

In certain embodiments, the end pH does not exceed 9.5, 9.4, 9.3, 9.2, 9.1, 9.0, 8.8, 8.9, 8.7, 8.6, 8.5, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, or 6.0. The end pH may range from 6.0 to 9.5.

Depending on the buffer used, the bioactive glass materials may be pretreated for different periods such that they become suitable for bone regeneration. Pre-treating the bioactive glass materials much longer than necessary to activate them may lead to deactivation. Similarly, if the bioactive glass materials are not pre-treated long enough, they may remain too active. In some embodiments, bioactive glass materials may be pretreated with the buffer for as short as 30 minutes. Other embodiments of the bioactive glass materials may require pretreatment as long as 24 hours. In some embodiments, the bioactive glass materials may be pretreated about 1 to about 2 hours, about 3 to about 4 hours, about 5 to about 6 hours, about 7 to about 8 hours, about 9 to about 10 hours, about 11 to about 12 hours, about 13 to about 14 hours, about 15 to about 16 hours, about 17 to about 18 hours, about 19 to about 20 hours, about 21 to about 22 hours, or about 23 to about 24 hours. Some bioactive glass materials may require pretreatments longer than 24 hours. As used here in the context of pre-treatment time, the term “about” means +/−30 minutes. A person skilled in the art can easily design simple experimental procedures to determine the optimum pretreatment time for any given bioactive glass material that is to be coated with a GAG. After pretreatment, the bioactive glass material may be dried before being coated with a GAG.

In another embodiment of this aspect, silicate ions are released into and/or onto the wound and/or bone defect. In yet another embodiment of this aspect, calcium ions are released into the bone. In a further embodiment, borate ions are released into the bone. In any of the above embodiments of this aspect of the invention, sufficient ions, which include but are not limited to silicate, calcium, and borate, are released from the bioactive glass ceramic into the bone defect to achieve a critical concentration of ions to stimulate the proliferation and differentiation of an osteoblast and/or are released into the tissue at the site of the wound to promote wound healing.

The bioactive glass particles, fibers, sheet or mesh can further comprise a carrier or a graft extender. The carrier may be one or more of hydroxyapatite, tricalcium phosphate, and calcium salts such as, but not limited to, calcium sulfate and calcium silicate. The bioactive glass may be in a granular form and comprise other materials as carriers as well.

In some embodiments, the bioactive glass fibers forming the bioactive glass sheet may be arranged in a variety of structures to form a wrap. In these structures, the bioactive glass fibers may be woven, knitted, warped knitted, and/or braided. The bioactive glass fibers can also form a mesh-like structure. The bioactive glass sheet may be formed such that it has a substantially greater length than width.

In any of the woven, knitted, warped knitted, or braided patterns, the bioactive glass fibers are in both a longitudinal and transverse orientation. For example, the longitudinal fibers may be interwoven with transverse fibers. Some transverse fibers can be wrapped around the outside of the longitudinal fibers to secure the longitudinal and transverse fibers. In one example, the transverse fibers may be wrapped around the longitudinal fibers to form a knot or whipping. Alternatively, the longitudinal fibers may be stitched to the transverse fibers.

The openings within the sheet or mesh may have a low density. The structure and density of the bioactive glass fibers may be similar to the density of material in VICRYL (Ethicon, Inc., Somerville, N.J.). The sheet or mesh may have any one or more of about 5%, 10%, 15%, 20%, 25%, 30%, 35%, 40%, 45%, 50%, 55%, 60%, 65%, 70%, 75%, 80%, 85%, 90%, and 95% empty opening as a percentage of the area. The density of the mesh or sheet may be sufficiently high, i.e. the openings must have a low enough percentage area, such that the wrap is able to be sutured. The density may also be high enough such that the wrap serves as a barrier to hyperplasis and tissue adhesion.

The bioactive glass particles, fibers, sheet or mesh may have binding regions, which may enable the mesh or sheet to be anchored to the bone. A bone anchoring device can affix or anchor the sheet or mesh by extending through the sheet or mesh. Exemplary bone anchoring devices include screws, sutures, stables, pins, buttons, and combinations thereof. The bone anchoring device can be attached to a drilled or hollowed out region of bone.

The bone defect may be a fracture or may result from an injury or bone disease, such as osteoporosis. The bioactive glass sheet may release one or more of silicate, calcium, and borate ions into the bone defect. Ions released into the bone defect can stimulate osteoblast activity. The bioactive glass sheet may further comprise a carrier, such as hydroxyapatite and tricalcium phosphate, or a graft extender.

The bioactive glass ceramic provided by this aspect of the invention may be effective to produce hydroxyapatite in a hard tissue and to promote wound healing. An exchange of ions may occur between bioactive glass and the surrounding body fluid that results in the production of hydroxyapatite. The exchanged ions may also enhance the rate of wound healing, such as by stimulating collagen production and/or by activating genes responsible for cell proliferation at the site of a wound. The ions exchanged may be any one or more of silicate, calcium, and borate.

Glycosaminoglycans (GAGs) are polysaccharides that are present in various cells. There are many different types of GAGs have been found in tissues and fluids of humans, animals, and other vertebrates. GAGs are typically linear molecules with greatly varying chain lengths composed of heterogeneous polysaccharides and are formed by long disaccharide units with varying degrees of linkage, acetylation, and sulfation. The disaccharide units include galactose, N-acetylglucosamine, N-acetylgalactosamine, and glucuronic acid. GAGs are often classified as being sulfated or non-sulfated.

Known GAGs have been classified as being one of chondroitin sulfate, keratan sulfate, dermatan sulfate, hyaluronic acid, heparin, and heparan sulfate. Along with collagen, GAGs provide significant structural support to animal tissue. Without GAGs, tissues would not undergo proper repair. Further, the protection and maintenance of all tissues depends upon GAGs. Thus, incorporation of GAGs onto the surface of bioactive glass can serve to provide support to wounded tissue, particularly in the context of wounded tissue at or near the site of a bone injury.

Further, GAGs on the surface of bioactive glass may even be helpful to heal wounds not associated with a bone injury because glass fibers may be useful for wound healing. See, U.S. Patent Publication No. 2011/0165221 to Jung et al. Local sources of calcium, magnesium, zinc, silica, borate, strontium, silver and other ions that may be included in and/or released by bioactive glass may enhance the rate of wound healing. The presence of additional calcium, magnesium, silica, sodium, borate and zinc ions, in particular, may serve to signal cells to enhance the rate of wound healing. Silica ions, along with the increased pH arising from release of sodium ions are conducive to wound healing. In addition, articles by Jung et al. indicate that borate ions may promote wound healing. Silver ions may be effective to reduce inflammation and to inhibit bacterial growth. With regard to bone repair, the presence of calcium and silica ions at critical concentrations near the bone can activate genes responsible for osteo progenitor cells to differentiate into osteoblasts. See, e.g., Jones, J. R. et al, “Extracellular matrix formation and mineralization on a phosphate-free porous bioactive glass scaffold using primary human osteoblast (HOB) cells” Biomaterials, 2007, 28(9):1653-63.

In some embodiments, the glycosaminoglycan is bound to the bioactive glass by means of an ionic bond. GAGs have hydroxyl groups, amide groups, and oxygen atoms that may form ionic bonds with positively-charged ions in bioactive glass. Such positively-charged ions include calcium, strontium, magnesium, potassium and sodium. Positively-charged ions also include divalent cations such as Ca²⁺, Cu²⁺, Mg²⁺, Fe²⁺, Fe³⁺, Sr²⁺, Cd²⁺, Al²⁺, Cr²⁺, Co²⁺, Mn²⁺, Ni²⁺, Sn²⁺, and Zn²⁺.

In some embodiments, the glycosaminoglycan is bound to the bioactive glass by means of a covalent bond. Glycosamionoglycans have carboxylic acid groups, hydroxyl groups, and amide functional groups that can be used to form covalent bonds with bioactive glass through the use of a silane coupling agent. An exemplary silane coupling agent is aminopropyl silane. Such coupling agents are available from Gelest Inc., for example. Exemplary coupling agents include, but are not limited to, aminopropyl triethoxysilane, diaminopropyl diethoxysilane, glycidoxypropyl trimethoxysilane, aminopropyl trimethoxysilane, and aminopropyl triethoxysilane. When these coupling agents are used, the silica group would covalently bind to the glass and the aminopropyl portion can form a covalent bond with the GAG. The end result would be a covalent linkage of the GAG to the bioactive glass.

In various embodiments of this and other aspects of the invention, the GAG need not remain ionically- or covalently-bound to the bioactive glass after implantation of a GAG-coated bioactive glass material into the body. In the body, the GAG may eventually be hydrolyzed from the bioactive glass. The bioactive glass and the GAG would both be present in the tissue near the implant site. Both substances can then promote healing of the wound at the implant site. Without being bound by any particular mechanism, the bioactive glass may promote bone repair and induce soft tissue repair by the release of calcium ions. The GAGs may promote angiogenesis, enhance soft tissue repair, reduce inflammation, and/or counteract any tendency of the bioactive glass in the wound site to promote coagulation.

In some embodiments, the glycosaminoglycan is heparin. Heparin is known to promote anticoagulation. Heparin is known to interfere with key molecules in the blood clotting mechanism to block blood clotting. Reduction of blood coagulation at the site of a wound may increase the rate at which the wound heals. Heparin may be particularly active in reducing coagulation when present on the surface of a prosthetic compound, such as a bioactive glass filament, sheet, or mesh.

Heparin may also serve as an anti-inflammatory. One proposed mechanism of heparin anti-inflammatory activity is through the blocking of histamine activity. Also, reduction of inflammation at the site of a wound may increase the rate at which the wound heals. Heparin may also promote wound healing by initiating angiogenesis. Increased blood flow may increase the rate of wound healing.

Heparin may also serve to protect wounds against invading bacteria and other pathogens as this glycosaminoglycan is released by mast cells at sites of injury. Reducing the numbers of bacteria and pathogens may serve to increase the rate of wound healing. In this way, the presence of heparin can further enhance the antibiotic activity of bioactive glass itself. Such enhancement may be synergistic.

In some embodiments, the glycosaminoglycan is heparan sulfate, which is found on numerous proteoglycans involved in wound healing. Heparan sulfate also may play a role in angiogenesis as mice deficient in perlecan heparin sulfate exhibit delayed wound healing and defective angiogenesis. See, Zhou et al., Cancer Res. (2004) 64:4699. Heparan sulfate coated bioactive glass may promote wound healing. For instance, the heparin sulfate may act to regulate basement membrane permeability, epidermal hyperproliferation, dermal fibrosis, cell attachment and migration, and growth factor binding.

In some embodiments, the glycosaminoglycan is chondroitin sulfate, which is found in bone, the corneum, and in cartilage. Chondroitin sulfates are often polysulfated and are named by the position of the sulfate group. For instance, chondroitin-4 sulfate is present in bones, flesh, and skin. Bioactive glasses coated with chondroitin sulfate, such as chondroitin-4 sulfate, may be useful in the treatment of osteoarthritis.

In some embodiments, the glycosaminoglycan is dermatan sulfate. Dermatan sulfate is present in the skin. Dermatan sulfate has historically been referred to as chondroitin sulfate B, even though it is no longer considered a chondroitin. As discussed by Trowbridge, J. M. and Gallo, R. L., Glycobiology, 2002, 12(9):117R-125R, dermatan sulfate may play a role in wound healing.

In some embodiments, the glycosaminoglycan is keratan sulfate. Keratan sulfate has two varieties (I and II) and are present throughout a wide variety of tissues, such as in the cartilage and loose connective tissues. A significant amount of keratan sulfate is found in the cornea. However, non-corneal keratan sulfate is found in bone and cartilage. In bone and cartilage, osteoadherin, fibromodlin and PRELP are proteins that are modified by N-linked keratan sulfate chains.

In some embodiments, the glycosaminoglycan is hyaluronic acid. Hyaluronic acid is a non-sulfated GAG and is typically not covalently attached to proteins as a proteoglycan. It is, however, a component of non-covalently formed complexes with proteoglycans in the ECM. Hyaluronic acid is known to produce viscous and thick solutions as hyaluronic acid polymers have molecular weights exceeding 100,000 amu and can displace a large volume of water. Hyaluronic acid-containing solutions are not readily compressible. This property makes them excellent lubricators and shock absorbers. Synovial liquids in joints, for instance, are often rich in hyaluronic acid.

Hyaluronic acid may also promote wound healing by filling in spaces in which cells could otherwise migrate. Space created by hyaluronic acid allows for enhanced cell migration, cell attachment, during wound repair, organogenesis, immune cell adhesion, activation of intracellular signaling, as well as tumor metastasis. These roles are mediated by specific protein and proteoglycan binding to this GAG. Hyaluronic acid may assist in forming proteoglycans, such as cartilage aggrecans. Hyalurionic acid may interact with CD44 receptors and other integrin. Bioactive glasses coated with hyaluronic acid may be useful in the treatment of osteoarthritis as there is often a deficiency of hyraluronic acid in osteoarthritic joints.

In any of the embodiments of this aspect, the composition promotes more rapid wound healing than that achieved by an uncoated bioactive glass particle. The GAGs present on the bioactive glass serve to enhance the rate of wound healing. Further, the ions released by the bioactive glass combined with the activity of the GAGs may synergistically enhance the rate of wound healing. Synergy may arise from any one or more of the following GAG activities: reduction of blood clotting and/or coagulation, facilitation of the migration of cells into the scaffold, formation of blood vessels, and stimulation of genes to increase the rate of healing of hard and soft tissues.

The invention also provides, in another aspect, for a material comprising collagen and bioactive glass, with the bioactive glass coated with a GAG. The GAG is bound to the bioactive glass. The bioactive glass may be in the form of a particle, a glass sheet, a fiber, a mesh, or any combination of these forms. The GAG can be any of the GAGs discussed above or known in the art. The collagen may be crosslinked to varying degrees.

Another aspect of the invention provides for a method for treating a wound. Bioactive glass coated with a glycosaminoglycan is applied to the wound. The bioactive glass may be in the form of a particle, a glass sheet, a fiber, a mesh, or other shape. The preparation of the particle, sheet, fiber, mesh or other shape may be undertaken as described above. The wound comprises one or more of a bone injury and a soft tissue injury. The coated bioactive glass is effective to accelerate repair of the bone injury and the soft tissue injury.

In some embodiments, the GAGs and the bioactive glass together reduce the amount of inflammation in the bone and/or surrounding soft tissue. Reduced inflammation may enhance the rate of both bone formation and soft tissue wound healing.

In some embodiments, the GAGs may reduce the amount of coagulation of blood and/or other cells in the tissue and vasculature of the wound. The wound may surround a bone defect. Reduced coagulation may enhance the rate of wound healing, particularly wounded soft tissue. Enhanced wound healing may also serve to enhance the rate of bone formation as inflammation is more quickly reduced in any surrounding soft tissue by the enhancement of wound healing.

In any of the embodiments of this aspect, the glycosaminoglycans on the bioactive glass may be any one or more of heparin, heparin sulfate, chondroitin sulfate, dermatan sulfate, keratan sulfate, and hyaluronic acid. These glycosaminoglycans may enhance the rate of wound healing and/or bone formation by at least the mechanisms discussed above with respect to each of the glycosaminoglycans. The enhancement of wound healing and/or bone formation may be synergistic.

Another aspect of the invention provides for a method of treating a bone defect. A bioactive glass coated with a glycosaminoglycan is applied to the site at or near the bone defect. The bioactive glass may be in the form of a particle, a glass sheet, a fiber, a mesh, or any combination of these forms. The coated bioactive glass is bioresorbable at a rate consistent with the rate of formation of new bone at or near the site.

In some embodiments, the coated bioactive glass compositions bioresorb at a rate approximately equivalent to the rate of formation of new bone at or near the site of the bone defect. Such bioresorption may result from a significant contribution of ions from the bioactive glass to the surrounding tissue and/or bone such that the ions are incorporated into the tissue and/or bone. It may also be the case in some embodiments that the rate of mass increase of the bone at or near the site of the bone defect is consistent with the rate at which the mass of the composition decreases. The rate of bioresportion may be controlled by altering the size of the bioactive glass particles and/or the composition of ions within the bioactive glass particles.

Another aspect of the invention provides for a method of stimulating the activity of a gene that promotes wound healing and/or bone regeneration. Bioactive glass coated with a glycosaminoglycan is applied to the site at or near the bone defect. The bioactive glass may be in the form of a particle, a glass sheet, a fiber, a mesh, or any combination of these forms. The activity of the gene is stimulated.

In any of the embodiments of this aspect, the glycosaminoglycans on the bioactive glass may be any one or more of heparin, heparin sulfate, chondroitin sulfate, dermatan sulfate, keratan sulfate, and hyaluronic acid.

In various embodiments of this aspect, the gene may be one or more of BMP-2, Runx2, Osterix, DIx5, TGF-beta, PDGF, VEGF, collagen I, ALP (alkaline phosphatase), bone sialoprotein, P1 NP (procollagen type 1 N-terminal propeptide), osteoponin, osteonectin, and osteocalcin.

BMP-2, also known as bone morphogenetic protein 2, is a member of the TGF-beta superfamily of proteins. Stimulation of BMP-2 activity, such as by stimulating the BMP-2 gene and/or protein expression, can lead to stimulation of bone production. BMP-2 stimulation may enhance the overall rate and extent of bone defect repair.

Runx2, also known as Runt-related transcription factor 2, is a transcription factor that is associated with osteoblast development and differentiation. Mutations in the Runx2 gene are associated with Cleidocranial dysostosis, a general skeletal condition. Stimulation of Runx2 activity, such as by stimulating the Runx2 gene and/or expression of its associated protein, can lead to stimulation of bone production. Runx2 stimulation may enhance osteoblast formation and activity, as well as the overall rate and extent of bone defect repair.

Osterix is a transcription factor that plays a role in osteoblast differentiation and bone formation. As discussed in Cao et al., Cancer Res., 2005, 65:1124-8, Osterix may play a role in osteoblast differentiation and tumor activity in osteosarcoma. Stimulation of Osterix activity, such as by stimulating the Osterix gene and/or protein expression, can lead to stimulation of bone production. Osterix stimulation may enhance the overall rate and extent of bone defect repair.

DLX-5 is a protein that is encoded by the homeobox transcription factor gene DLX5. Mutations in DLX-5 may be associated with hand and foot malformations. Stimulation of DLX-5 protein expression and/or activity, as well as stimulation of DLX5 gene expression, may lead to stimulation of bone production and enhancement of bone defect repair.

TGF-beta (transforming growth factor beta) is a protein that exists in three isoforms, TGF-beta1, TGF-beta2, and TGF-beta3. Genes encoding these proteins include TGFB1, TGFB2, and TGFB3. Activation of these genes, as well as enhancement of the activity of the TGF-beta proteins, can promote tissue remodeling. Increased tissue remodeling can serve to enhance the rate of tissue repair and wound healing.

PDGF (platelet-derived growth factor) is a growth factor that regulates cell growth and division. PDGF plays a major role in angiogenesis, as well as cell proliferation, cell migration, and embryonic development. GAGs may serve to increase PDGF activity as a means to promote wound healing by any one or more of these mechanisms. PDGF is found as four ligands, PDGFA, PDGFB, PDGFC, and PDGFD. These ligands may form dimers. Also, PDGFA and PDGFB may form a heterodimer.

VEGF (vascular endothelial growth factor) is a family of growth factors that include VEGF-A, VEGF-B, VEGF-C, VEGF-D, and PGF (placenta growth factor). VEGF stimulates angiogenesis and promotes cell migration, both processes useful in the repair of soft-tissue wounds. GAGs may promote VEGF-mediated activity. Further, in various embodiments of any aspect of the invention, drugs such as bevacizumab and ranibizumab, which enhance VEGF activity, may be included in the GAG-bioactive glass compositions.

Collagen I, also known as type-I collagen, is found both in scar tissue and in the organic part of bone. Collagen I is also found in tendons and the endocmysium of myofibrils. Stimulation of collagen I production, such as by stimulating expression of genes associated with collagen I, including COL1A1 and COL1A2, may enhance the overall rate and extent of bone defect repair.

ALP, also known as ALKP and alkaline phosphatase, removes phosphate groups from many types of molecules. ALPL, an alkaline phosphatase isozyme, is found in various tissues of the human body, including bone. Stimulation of ALP and/or ALPL activity, may lead to stimulation of bone production. ALP and/or ALPL stimulation may enhance the overall rate and extent of bone defect repair.

Bone sialoprotein, also known as BSP, cell-binding sialoprotein or integrin-binding sialoprotein, is a significant component of bone extracellular matrix. The IBSP gene encodes bone sialoprotein. Stimulation of IBSP gene expression and/or bone sialoprotein expression, may enhance the overall rate and extent of bone defect repair. For example, bone sialoprotein could improve the mineralization of newly-formed bone matrix at the repair site.

Procallagen type 1 N-terminal propeptide, also known as P1 NP, is an effective marker of bone formation as this gene promotes collagen turnover. P1 NP expression is proportional to the amount of new collagen laid down when bone is formed. Stimulation of P1 NP gene expression and/or P1 NP protein expression, may enhance the overall rate and extent of bone defect repair by enhancing the rate of collagen deposition in the bone.

Osteopontin, also known as BSP-1, ETA-1, SPP1, 2ar, and Ric, is a protein expressed in bone, as well as other tissues. Ostepontin is synthesed by fibroblasts, preosteoblasts, osteoblasts, osteocytes, bone marrow cells, and endothelial cells. Osteopontin is known to be important in bone remodeling, such as by anchoring osteoclasts to the bone mineral matrix. Stimulation of osteopontin gene expression and/or osteopontin protein expression, may enhance the overall rate and extent of bone defect repair by enhancing the rate of bone formation.

Osteonectin, also known as SPARC or BM-40, is a protein encoded by the SPARC gene. Osteonectin binds sodium and is secreted by osteoblasts during bone formation. Osteonectin is thought to play an important role in bone mineralization and collagen binding. As high levels of osteonectin are detected in active osteoblasts, stimulation of SPARC gene expression and/or osteonectin protein expression may enhance the overall rate and extent of bone defect repair by enhancing the rate of bone formation.

Osteocalcin, also known as BGLAP, is a bone protein encoded by the BGLAP gene. Osteocalcin is secreted by osteoblasts and may play a role in bone mineralization. Stimulation of osteocalcin protein expression and/or BGLAP gene expression may enhance the overall rate and extent of bone defect repair.

Another aspect of the invention provides for a method of reducing inflammation at or near the site of a wound and/or a bone defect. Bioactive glass coated with a glycosaminoglycan is applied to the site of the wound or at or near the bone defect.

In some aspects, a compound bone fracture may be treated. A bone at the site of the compound bone fracture is wrapped with any of the above-described compositions of bioactive glass coated with glycosaminoglycans. The bioactive glass ceramic may be in the form of fibers, a fiber mesh, and a sheet. The compositions may have enhanced anti-inflammatory activities that serve to reduce pain and discomfort in the surrounding wounded tissue as the compound bone fracture heals.

The coated bioactive glass fibers, meshes, and sheets may be wrapped completely around the bone such that the ceramic is secured to the bone and/or maintains the bone shape so as to prevent further fracturing. One exemplary form of the bioactive glass ceramic is in the form of a mesh that can be wrapped around a large portion of bone surrounding the compound fracture so as to both provide pressure to the bone and to allow for the migration of ions from the mesh wrap into the bone. The bioactive glass ceramic may also be secured to the bone by one or more plates and/or one or more screws.

In any of the embodiments of this aspect, the glycosaminoglycans on the bioactive glass may be any one or more of heparin, heparin sulfate, chondroitin sulfate, dermatan sulfate, keratan sulfate, and hyaluronic acid.

Another aspect of the invention provides for a method of preparing a composition comprising bioactive glass coated with a glycosaminoglycan. The glycosaminoglycan is applied to the bioactive glass by means of dipping or spraying. The bioactive glass with glycosaminoglycan is then freeze-dried such that the glycosaminoglycan becomes attached to the bioactive glass by a physical bond.

The physical bond may be an ionic interaction. The ionic interaction may be between at least one negatively-charged group on the glycosaminoglycan and at least one positively charged ion within the bioactive glass. The negatively-charged groups may arise from hydroxyl groups, amide groups, and/or oxygen atoms on the glycosaminoglycan.

The physical bond may also be covalent. An aminopropyl silane coupling agent may be used to bring about the covalent bond, for example, aminopropyl trimethoxysilane and aminopropyl triethoxysilane. Such coupling agents are available from Gelest Inc., for example. The silica group may covalently bind to the glass and the aminopropyl portion may form a covalent bond with the GAG, resulting in a covalent linkage of the GAG to the bioactive glass.

One process for making the composition comprising bioactive glass coated with a glycosaminoglycan is by solubilizing the GAG's to form a 0.1-10% solution. The solution can be spray applied or poured onto/over the glass particles, fibers, sheets etc. Porous blocks of bioactive glass can be dipped into a GAG solution. The glass can then be dried using a variety of techniques, including but not limited to freeze drying, vacuum drying, oven drying, and spray drying. The process can be repeated until the desired ratio of GAG to glass is achieved.

Another process for making the composition is to prepare a sol-gel solution and electrospin fibers using a process similar to the one described by Kim et al., c 2006 Wiley Periodicals, Inc. J. Biomed. Mater. Res. 79A: 698-705, 2006. Essentially, the electrospun fibers are immersed in a GAG solution and pressed to form a sheet or freeze dried to form a porous scaffold.

Another process for making the composition is to prepare a sol-gel solution and GAG solution followed by simultaneous electrospinning of both materials into fibers using the process for making fibers which is similar to the one described by Kim et al., c 2006 Wiley Periodicals, Inc. J. Biomed. Mater. Res. 79A: 698-705, 2006. The electrospun fibers can be used in their cotton-like form or woven or pressed to form a sheet.

Another process for making the composition is to prepare a sol-gel solution which also contains GAG and electrospin fibers using the process for preparing fibers described by Kim et al., c 2006 Wiley Periodicals, Inc. J Biomed Mater Res 79A: 698-705, 2006. The electrospun fibers can be used in their cotton-like form or woven or pressed to form a sheet.

In various embodiments of this and other aspects of the invention, the GAG need not remain ionically or covalently bound to the bioactive glass after implantation of a GAG-coated bioactive glass material into the body. In the body, the GAG may eventually be hydrolyzed from the bioactive glass. The bioactive glass and the GAG would both be present in the tissue near the implant site. Both substances can then promote healing of the wound at the implant site. Without being bound by any particular mechanism, the bioactive glass may promote bone repair and induce soft tissue repair by the release of calcium ions. The GAGs may promote angiogenesis, enhance soft tissue repair, reduce inflammation, and/or counteract any tendency of the bioactive glass in the wound site to promote coagulation.

In some embodiments of any of the above aspects of the invention, the bioactive glass compositions may contain radioactive materials. Such bioactive glass compositions may be useful to treat tumors and bone defects arising out of cancer. The radiation emitted from the bioactive glass composition is effective to kill cancer cells within the tumors and bone defects.

Yet another aspect of the invention provides for a method of preparing a composition comprising bioactive glass coated with glycosaminoglycan fibers. A sol-gel solution comprising GAGs is electrospinned. The electrospinning may result in the formation of GAG fibers. The GAG fibers may be very fine, such as microfibers or nanofibers. The electrospinning may be conducted in the presence of bioactive glass particles dispersed within the solution. Alternatively, the bioactive glass particles can be incubated in the post-electrospun sol-gel solution comprising GAGs. The bioactive glass may be in the form of an interpenetrating network of bioglass. The method may result in the deposition of GAG fibers, such as GAG microfibers and GAG nanofibers, on bioactive glass particles.

Another aspect of the invention provides a method of preparing bioactive glass fibers, and use of GAGs as an electrospinning polymer. A sol-gel solution comprising GAGs is electrospinned. The electrospinning may result in the formation of GAG-bioglass fibers. The GAG-bioglass fibers may be very fine, such as microfibers or nanofibers. Alternatively, the GAG-bioactive glass fibers can be further incubated in a GAG solution. As another alternative, GAG solution can be sprayed on the resulting GAG-bioactive glass material. The bioactive glass may be in the form of an interpenetrating network of bioglass. The method may result in the deposition of GAG fibers, such as GAG microfibers and GAG nanofibers, on bioactive glass particles.

Compositions in accordance with the disclosure may also be sterilized by, for example, aseptic processing and ethylene oxide sterilization.

### EXAMPLES

### Example 1

### Preparation of Bioactive Glass Particles

40 grams of quartz silica, 20 grams of phosphorous pentoxide, 3 grams of calcium carbonate, and 3 grams of sodium carbonate is mixed together. The mixture is then placed in a platinum crucible and melted at 1440° C. for 1.5 hours and poured into demineralized water to produce a granular glass frit. The bioactive glass frit is dried and ground in a mill to produce a powder. The powder is sieved through a 0.2 micron mesh sieve.

### Example 2

### Preparation of Bioactive Glass Fibers

The above ceramic is further treated by pulling molten ceramic into fibers. The fibers are then mixed together and pressed to form a mesh.

A bioactive glass ceramic prepared above is made into fibers by spinning. In spinning, heating treatment steps are performed, followed by mechanical pulling of the fibers. Techniques for spinning and pulling fibers are well known.

The fibers are tested by immersing them in Tris for three, five and seven days, respectively. Clear precipitation of calcium phosphate occurs after three days. The precipitation occurs as large flakes and starts to decay at seven days. The precipitations are evenly distributed at the surface of the fibers and this shows the high uniformity of the material.

### Example 3

### Weaving of Bioactive Glass Fibers to Form a Mesh

Bundles of bioactive glass fibers are prepared by manually weaving the fibers into a simple biaxial pattern. Specific procedures for weaving different patterns are numerous and widely available to those skilled in the art. The woven mesh is cleaned in isopropanol and dried in air. The micropore size and the distance between bundles are in the range of 150-200 μm and 400-800 μm.

### Example 4

### Preparation of Glycosaminoglycan-Coated Bioactive Glass Particles

Hyaluronic acid is prepared in the form of solutions ranging in between 0.1-10%. The bioactive glass particles prepared according to Example 1 are then laid out slowly onto a bed. Solutions ranging in between 0.1-10% are then added to the bed of bioactive glass particles. The mixtures are then rocked gently for 24 hours so as to disperse the hyaluronic acid evenly throughout the bioactive glass particles. The bioactive glass particles are then isolated from the solution and allowed to dry.

### Example 5

### Preparation of Glycosaminoglycan-Coated Bioactive Glass Fibers

Chondroitin is prepared in the form of solutions ranging in between 0.1-10%. The bioactive glass fibers prepared according to Example 2 are then laid out slowly onto a bed. The solutions ranging in between 0.1-10% of chondroitin are then added to the bed of bioactive glass fibers. The mixture is then rocked gently for 24 hours so as to disperse the chondroitin evenly throughout the bioactive glass particles. The bioactive glass fibers are then isolated from the solution and allowed to dry.

### Example 6

### Preparation of Glycosaminoglycan-Coated Bioactive Glass Mesh

Bioactive glass fibers are prepared according to Example 2. The fibers are then woven into a mesh according to Example 3. Hyaluronic acid is prepared in the form of solutions ranging in between 0.1-10%. The bioactive glass mesh prepared according to Example 3 is then placed in a container and covered with the solutions ranging in between 0.1-10% of chondroitin. The container is gently agitated for 24 hours so as to disperse the hyaluronic acid evenly throughout the bioactive glass particles. The bioactive glass mesh is then isolated from the solution and allowed to dry.

### Example 7

### Testing of Bioactive Glass Ceramic In Vitro

The bioactive glass coated with hyaluronic acid prepared above is tested by mixing 0.075 g of the powder with 50 mL of simulated body fluid. A layer of calcium carbonated apatite forms on the surface in six hours, as confirmed by X-ray powder diffraction and Fourier Transform Infrared Spectroscopy.

### Example 8

### In Vivo Testing of Bone Graft Materials

Guinea pigs are prepared for surgery by removing the hair from the dorsum and wiping the area with povidone-iodine followed by 70% isopropyl alcohol. Four 1-cm incisions (2 on right side and 2 on left side) ˜1 cm from the midline were made through the skin on the dorsum to expose the underlying lumbar fascia. Using a tissue punch, a 6-8 mm diameter defect is created in the fascia and muscle layers at each incision site. The depth of the punch is ˜4 mm which penetrates through the fascia into the latissimus dorsi muscle, and into (not through) the erector spinae muscle layer. A scalpel is used to remove tissue within the area of the defect created by the biopsy punch. Each defect is placed sufficiently to minimize the incidence of test and control contacting one another. The incision site for each defect is then closed using non-resorbable suture. Photographs are taken of the test and control devices prior to surgery, during and periodically after surgical implantation. After necropsy the implanted tissue is evaluated histologically.

### Example 9

Bioactive glass fibers coated with hyraluronic acid are tested, with uncoated bioactive glass fibers serving as a control. The test and control material are implanted into two bilateral drill defects that are surgically created in the cancellous bone of the proximal humerus of 18 canines. A test material is implanted into one of the drill defects in the first humerus, while a control article is implanted into the other drill defect in the opposite humerus. The drill defects will be approximately 10 mm in diameter and approximately 25 mm in depth.

Animals will be sacrificed and the implantation sites harvested and analyzed at various predetermined time points. Analysis includes mechanical testing to assess the bony ingrowth and remodeling of the defect site. The extent of bone remodeling and healing within the drill defects are characterized by the histopathological and histomorphometry evaluation. Defects treated with the hyaluronic acid-coated bioactive glass fibers show improved healing at each of the predetermined time points.

### Example 10

### Clinical Testing of Bone Graft Materials

Bioactive glass fibers coated with hyraluronic acid are tested, with uncoated bioactive glass fibers serving as a control. Graft material that does not contain bioactive glass will serve as control. The test and control material are implanted into two bilateral drill defects that are surgically created, one defect on the left side of the iliac crest and one defect on the right side of the iliac crest. The test material is planted into the left iliac crest and the control material was planted into the right iliac crest defect. At 12 weeks, all of the bone defects treated with test material had healed in each of the 25 subjects. At 15 weeks, 18 of the 25 bone defects treated with control material had healed in the 25 subjects.

### Example 11

### HA-BG (Hyaluronic Acid-Bioglass) Putty in Accordance with Description

HA-BG Putty Composition Formulation

An acceptable putty composition was be formulated by mixing the following ingredients. The formulation had a total glass % varying between 80-97% (BG: BG+HA, w:w) which was good and acceptable. The glass size varied between, 1 μm to 5 mm and was hydrated with saline or blood. The formulated putties were moldable and irrigation resistant.

### Example 12

### Composite HA-BG Formulation in Accordance with the Description

HA-BG Composite Composition Formulation

An acceptable HA-BG composite composition may be formulated by mixing the following ingredients. The formulation has a total glass % varying between 80-97% (BG: BG+HA, w:w) which is good and acceptable. The glass size varied between, 1 μm to 5 mm. The formulated putties are moldable and irrigation resistant. They were then hydrated with RODI (Reverse Osmosis Deionized water) and lyophilized. The composite may be hydrated with saline or blood.

### Example 13

### HA-BG Composite Composition Formulation II in Accordance with the Description

HA-BG Composite Composition Formulation II

An acceptable BG-HA composite composition may be formulated by mixing the following ingredients. HA was solubilized within 0.05-10% (w:w) RODI. The formulation has a total glass % varying between 80-97% (BG: BG+HA, w:w). The glass size varies between, 1 μm to 5 mm. the mixture is poured into the molds and then lyophilized. The composites are hydrated with saline or blood prior to use. The formulated putties are moldable and irrigation resistant.

### Example 14

### Injectable HA-BG Composition in Accordance with the Description

Acceptable Injectable HA-BG Composition Formulation

An acceptable flowable putty composition was formulated by mixing the following ingredients. HA was solubilized from 0.05-10% (w:w). The formulation had a total glass % varying between 1-97% (BG: BG+HA, w:w). The glass size varied between, 1 μm to 5 mm. The mixture was injectable though a syringe.

### Example 15

### Sterilization Example

Putty Processing with Sterilization

Appropriate amounts of Bioglass and Hyaluronic Acid were weighed on an analytical balance. These components were mixed then packaged for sterilization. Samples were Gamma Ray sterilized, E beam sterilized, EO sterilized, or remained unsterilized.

TABLE 1
Components of HA Bioglass Putty

| 1-2 mm | 710-90 μ | 32-125 μ | |
|---|---|---|---|
| BG | BG | BG | HA |
| 63.50% | 15.75% | 15.75% | 5% |
| 6.35 g | 1.575 g | 1.575 g | 0.5 g |

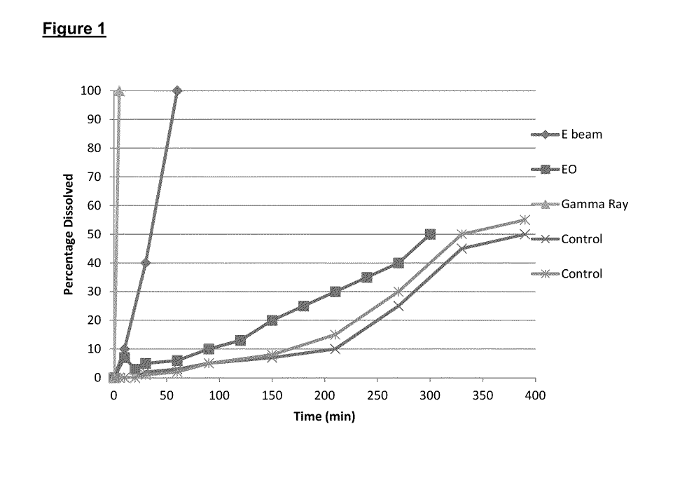

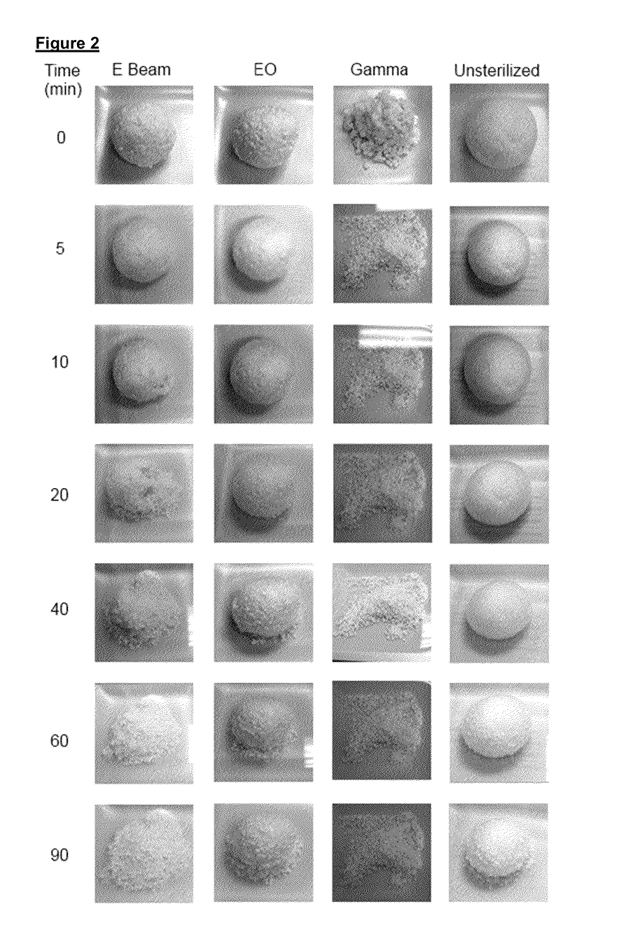

After sterilization, 3 to 3.5 mL of RODI was mixed into the components to make a small ball of putty. The percentage of dissolution was recorded after 5, 10, 20, 40, 60, 90, 150, 210, 270, 330, 390 and 450 minutes, or until all samples were completely dissolved. Gamma Ray sterilized samples completely dissolved after 5 minutes of immersion, thus dissolving at a rate of 20% per minute. E Beam sterilized samples were 100% dissolved after 60 minutes, thus dissolving at a rate of 1.59% per minute. EO sterilized samples dissolved slower than E Beam and gamma irradiation, with 50% of the sample being dissolved in 300 minutes, thus dissolving at a rate of 0.15% per minute. Unsterilized samples (control samples) were 52.5% dissolved in 390 minutes, thus dissolving at a rate of 0.12% per minute. The results are plotted in FIG. 1 and FIG. 2 includes photomicrographs of putties prepared by each sterilization method and unsterilized control described in this Example after 0, 5, 10, 20, 40, 60 and 90 minutes immersion in RODI.

Throughout this specification various indications have been given as to preferred and alternative embodiments of the invention. However, the foregoing detailed description is to be regarded as illustrative rather than limiting and the invention is not limited to any one of the provided embodiments. It should be understood that it is the appended claims, including all equivalents, that are intended to define the spirit and scope of this invention.

1. One or more particles of bioactive glass coated with a glycosaminoglycan, wherein the glycosaminoglycan is bound to the bioactive glass.

2. The composition of claim 1, wherein the glycosaminoglycan is bound to the bioactive glass by means of an ionic bond.

3. The composition of claim 1, wherein the glycosaminoglycan is bound to the bioactive glass by means of a covalent bond.

4. One or more bioactive glass sheets, fibers, or meshes coated with a glycosaminoglycan, wherein the glycosaminoglycan is bound to the bioactive glass.

5. The composition of claim 4, wherein the glycosaminoglycan is bound to the bioactive glass sheets, fibers, or meshes by means of an ionic bond.

6. The composition of claim 4, wherein the glycosaminoglycan is bound to the bioactive glass sheets, fibers, or meshes by means of a covalent bond.

7. The composition of any one of claim 1, wherein the glycosaminoglycan is heparin.

8. The composition of claim 1, wherein the glycosaminoglycan is heparan sulfate.

9. The composition of claim 1, wherein the glycosaminoglycan is chondroitin sulfate.

10. The composition of claim 1, wherein the glycosaminoglycan is dermatan sulfate.

11. The composition of claim 1, wherein the glycosaminoglycan is keratan sulfate.

12. The composition of claim 1, wherein the glycosaminoglycan is hyaluronic acid.

13. The composition of claim 1, wherein the composition promotes more rapid wound healing than that achieved by an uncoated bioactive glass particle.

14. A method for treating a wound comprising
applying a composition of claim 1 to the wound,
wherein the wound comprises one or more of a bone injury and a soft tissue injury; and
wherein the composition is effective to accelerate repair of the bone injury and the soft tissue injury.

15. The method of claim 14, wherein the composition is effective to reduce inflammation at the site of soft tissue injury.

16. The method of claim 14, wherein the composition is effective to reduce coagulation of blood and/or other cells at the site of the soft tissue injury.

17. The method of claim 14, wherein the glycosaminoglycan is heparin.

18. The method of claim 14, wherein the glycosaminoglycan is heparan sulfate.

19. The method of claim 14, wherein the glycosaminoglycan is chondroitin sulfate.

20. The method of claim 14, wherein the glycosaminoglycan is dermatan sulfate.

21. The method of claim 14, wherein the glycosaminoglycan is keratan sulfate.

22. The method of claim 14, wherein the glycosaminoglycan is hyaluronic acid.

23. The method of claim 14, wherein the composition increases the rate of wound healing, as compared to either glycosaminoglycan or bioactive glass alone.
